(19) United States
(12) Patent Application Publication
(10) Pub. No.: US 2019/0105058 A1
(43) Pub. Date: Apr. 11, 2019

(54) CUTTING TEMPLATE AND A METHOD OF MANUFACTURING THE SAME

(71) Applicant: UNIVERSITÄT BERN, Bern (CH)

(72) Inventor: Matthias MOTTINI, Muri (CH)

(73) Assignee: UNIVERSITÄT BERN, Bern (CH)

(21) Appl. No.: 16/086,552

(22) PCT Filed: Mar. 9, 2017
(86) PCT No.: PCT/EP2017/055576

§ 371 Date: Sep. 19, 2018

(30) Foreign Application Priority Data
Mar. 20, 2016 (EP) 16161300.5

(51) Int. Cl. A61B 17/15; A61B 34/10; A61B 17/17

(52) U.S. Cl. CPC A61B 17/15; A61B 2034/108; A61B 17/176; A61B 34/10

(57) ABSTRACT

A cutting template for mandibular surgery is described. The cutting template comprises a base; at least two cutting guides for guiding a bone cutting element for cutting a bone, and supported by the base; at least two openings for receiving bone fixation means for fixing the cutting template to the bone; and at least one positioning element for positioning the cutting template against the bone, and comprising a bone mating surface shaped with the contour of the bone against which it is designed to be fitted, and arranged to be in intimate contact with the bone during cutting.

### FIELD OF THE INVENTION

The invention relates to the field of cutting templates for cutting a bone, such as a mandible or fibular flap. The invention also relates to a method of manufacturing the cutting template and to a computer program product comprising instructions for implementing the steps of the method.

### BACKGROUND OF THE INVENTION

Bone reconstructions are often challenging task for surgeons. For instance, reconstruction of the oromandibular complex is often considered as a major challenge for maxillofacial surgeons, because of the complex anatomy, the sensitivity of the involved systems and the facial appearance. The mandible plays an important role in providing structural support for the floor of the mouth, tongue, and lips. It also provides the mobile bony platform for the teeth, allowing chewing. Surgical interventions to control cancer may result in a loss of mandibular continuity and the creation of soft tissue defects. As a result, these reconstructions aim to restore the above functionalities and re-establish facial symmetry and harmony as far as possible for various pathologies such as tumour-related deformities.

Multiple surgical techniques and materials, including autograft, allograft, allogeneic and xenograft products, have been used to perform such reconstructions. However, autogenous bone is the only graft material with osteoconductive, osteoinductive, and osteogenic potential. Therefore, in recent decades, the fibula free flap has been frequently used for instance for mandibular reconstructions, because it provides sufficient bone for large mandibular defects, a skin paddle for soft tissue demands, and adequate pedicle length with endosteal and periosteal blood supply conducive to multiple osteotomies of the fibula. In addition, its distance from the head and neck allows a simultaneous two-team approach with low donor site morbidity. However, good bone-to-bone contact between the fibula segments is difficult to achieve, and in many approaches, the tightening of the bone screws may cause the replacement bone segments to move outwards and away from one another, for example. The problem of contouring the straight fibula flap to resemble a section of mandible has been addressed in a variety of ways. Because of the complex 3-dimensional anatomy of the skull and face, this kind of operation usually requires extensive and expensive pre-surgical planning. The most common method of pre-operative virtual modelling of the patient's skull is three-dimensional (3D) representation and simulation using volumetric image datasets created from pre-operative computed tomography scans, which allow precise pre-operative planning and virtual reconstruction to be carried out using various software or freeware applications.

The goal of these new technologies in the field of cranio-maxillofacial surgery is to increase the predictability and precision of the planned operation, as well as to decrease morbidity, time of ischemia of the flap, and the overall operation time, thereby reducing costs and length of hospitalisation, and improving quality of life. However, these planning solutions and their associated tools are typically operated by specialist external engineers and require expensive and time-consuming face-to-face consultation or interactive online meetings with the surgeons to prepare the reconstruction operations. Also, prior art operation-planning arrangements tend to result in sub-optimal cutting templates (the guide devices used for the bone cutting), which do not enable an accurate cutting operation. Inaccurately cut bone segments in turn result in poor bone reconstruction and additional post-operative problems. Furthermore, prior art template manufacturing methods are complex and may result in a template which is not satisfactory in terms of cutting accuracy and speed.

There is thus a need for an improved cutting template and its manufacturing process for use in bone cutting operations, which enables a more accurate, reliable and quick bone cutting operation.

### BRIEF DESCRIPTION OF THE INVENTION

The present invention aims to overcome at least some of the disadvantages of prior art templates, and to meet the objectives described above. To this end, a cutting template according to the invention is described in the attached claim 1. The proposed cutting template has the advantage that it can be fixed directly to the deperiostated bone to be cut. As a result, a highly accurate cut can be achieved using the cutting guides of the template. The proposed cutting template enables for instance an extensive mandibular reconstruction to be carried out using the fibula free flap, with the help of a virtual preoperative 3-dimensional modelling method, in which all the necessary steps can be performed by the surgeon without the need for consultation or intervention by the technicians who have hitherto been required to produce and manage the template. This greatly reduces the costs and time required for the operation, including the planning and preoperative phases, for example with prosthetic dentists, compared to prior art methods. The invention also relates to a method of manufacturing the cutting template and to a computer program product comprising instructions for implementing the steps of the method, as recited in the attached claims.

### BRIEF DESCRIPTION OF THE DRAWINGS

The invention will now be described in more detail with reference to the attached drawings, in which:

FIG. 1 shows a schematic, isometric view of an example of a cutting template according to the invention.

FIG. 2 shows a schematic, isometric view of the cutting template of FIG. 1 together with a part of a fibular flap.

FIG. 3 shows a schematic, isometric view of another example of a cutting template according to the invention.

FIG. 3 shows a flow chart of an example method of manufacturing the cutting template according to the invention.

FIG. 4 shows a schematic, isometric view of a computer-generated model of a human mandible.

FIG. 5 shows a schematic, isometric view of the model of FIG. 5, where part of the mandible has been replaced with replacement bone structures.

FIG. 6 shows a schematic, isometric view of a manufactured model of a reconstructed mandible, for pre-fitting a bridging reconstruction plate.

FIG. 7 shows a schematic, isometric view of another example of a cutting template according to the invention.

### DETAILED DESCRIPTION OF THE INVENTION

It should be noted that the figures are provided merely as an aid to understanding the principles underlying the invention, and should not be taken as limiting the scope of protection sought. Where the same reference numbers are used in different figures, these are intended to indicate similar or corresponding features. It should not be assumed, however, that the use of different reference numbers is intended to indicate any particular degree of difference between the features to which they refer. The present invention will be explained in the context of apparatuses used for mandibular reconstruction with the use of bone parts from the fibular free flap. However, it is to be noted that teachings of the invention are not restricted to be used for mandibular reconstructions with the use of bone parts from the fibular flap. More generally, the teachings of the invention can be applied to the reconstruction of any bone parts with the use of any suitable replacement bone parts.

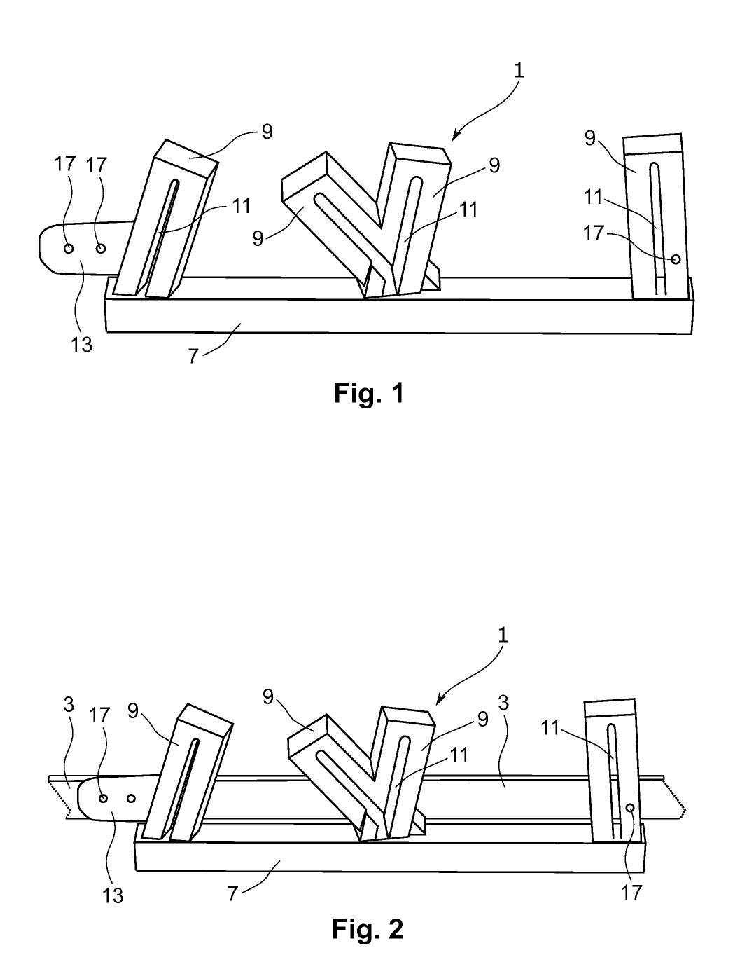

FIG. 1 illustrates an example cutting template 1 according to an embodiment of the present invention. This example cutting template 1 is designed to cut the fibular free flap (indicated by reference sign 3 in FIG. 2) so that excised sections of the fibula can be used in the mandibular reconstruction. The structural details of the cutting template 1 are explained first, followed by a description of an example method of manufacturing the cutting template. The example cutting template 1 comprises a base or support 7, which is designed to support cutting guides 9 protruding from the elongated base 7. The base 7 of the example cutting template 1 of FIGS. 1 and 2 is substantially straight, and the cutting guides 9 extend substantially in a common plane with the base 7. In this manner, the whole cutting template 1 is substantially planar, which is advantageous when the template 1 is generated using 3D printing, because the amount of time and material required for the printing process can be greatly reduced.

As shown in FIGS. 1 and 2, the cutting template 1 comprises four cutting guides 9. With the cutting template 1 of FIGS. 1 and 2, it is possible to cut two separate bone portions from the fibular flap to be used for the mandibular reconstruction. As shown, each cutting guide 9 has a longitudinal opening 11 or aperture, also referred to as a cutting slit or slot, for a bone saw to pass through. The cutting slots 11 each define a cutting plane, which may have a unique orientation with respect to the base 7. Each cutting slot 11 thus defines a precise cutting angle with respect to the bone to be cut. This cutting angle may be defined by at least two different rotational degrees of freedom. The cutting planes defined by different cutting slots may or may not be parallel to each other.

The cutting template 1 also comprises at least one positioning element 13, which in this example is a longitudinal element extending substantially in the plane of the base 7. In the example of FIGS. 1 and 2, the positioning element 13 is connected to the lefthandmost cutting guide 9. It is to be noted that, according to another example, the bone-facing surface of the cutting guide 9 may constitute the positioning element 13. This element 13 is used to precisely position the cutting template 1 against a deperiostated bone surface, in this description the surface of the fibular flap 3. The bone-facing side of the positioning element 13 in this example comprises a bone mating surface shaped with the contour of the bone against which it is designed to be fitted. In this manner the cutting element is designed to be in intimate contact with the hard deperiostated surface of the bone during the bone cutting operation. The cutting template 1 may comprise more than one positioning element 13. For example, referring to FIGS. 1 and 2, another positioning element could be connected to the righthandmost cutting guide 9 and extend to the right. The positioning elements 13 are preferably secured, for example using bone screws, to the stump of the bone, ie to the healthy part of the bone which is not to be cut away. In this way, a sound fixing of the cutting template can be achieved. Alternatively, the positioning elements 13 can be configured such that they extend for example towards the central cutting guides 9. In this manner the design of the cutting template may be made be more compact.

FIG. 2 shows schematically a portion of the fibular flap 3, with the cutting template 1 in its cutting position. The cutting template 1 also comprises holes 17 or openings for fixation means, such as bone screws, to pass through, so that the cutting template 1 can be fixed to the bone. In the illustrated example, the positioning element 13 is provided with two holes 17, and the right-hand cutting guide is provided with one fixing hole 17. Any or each of the cutting guides 9 may comprise one or more fixing holes 17. The cutting guides 9 are preferably shaped so that they are in direct contact with the bone during the bone cutting operation. Some or each of the cutting guides 9 may thus have a bone-mating surface, precisely shaped with the contour of the bone against which it is designed to be fitted. In this manner, it can be ensured that the cutting template 1, and more specifically the cutting guides 9, are in firm contact with the bone during the bone cutting operation.

The example cutting template 1′ of FIG. 7 can be used to cut the mandible, for example. This example cutting template 1′ comprises a first part and a second part. Each of the first and second parts comprises one cutting guide 9′ to guide the bone saw. In this example cutting template 1′, the bone-facing surfaces of the first and second parts may constitute the positioning element, which is used to precisely position the cutting template 1′ against a deperiostated bone surface, in this description the surface of the mandible. The bone-facing side of the positioning element 13 in this example comprises a bone mating surface shaped with the contour of the bone against which it is designed to be fitted. In this manner the cutting element is designed to be in intimate contact with the hard deperiostated surface of the bone during the bone cutting operation. The cutting template 1′ also comprises holes 17 or openings for fixation means, such as bone screws, to pass through, so that the cutting template 1′ can be fixed to the bone.

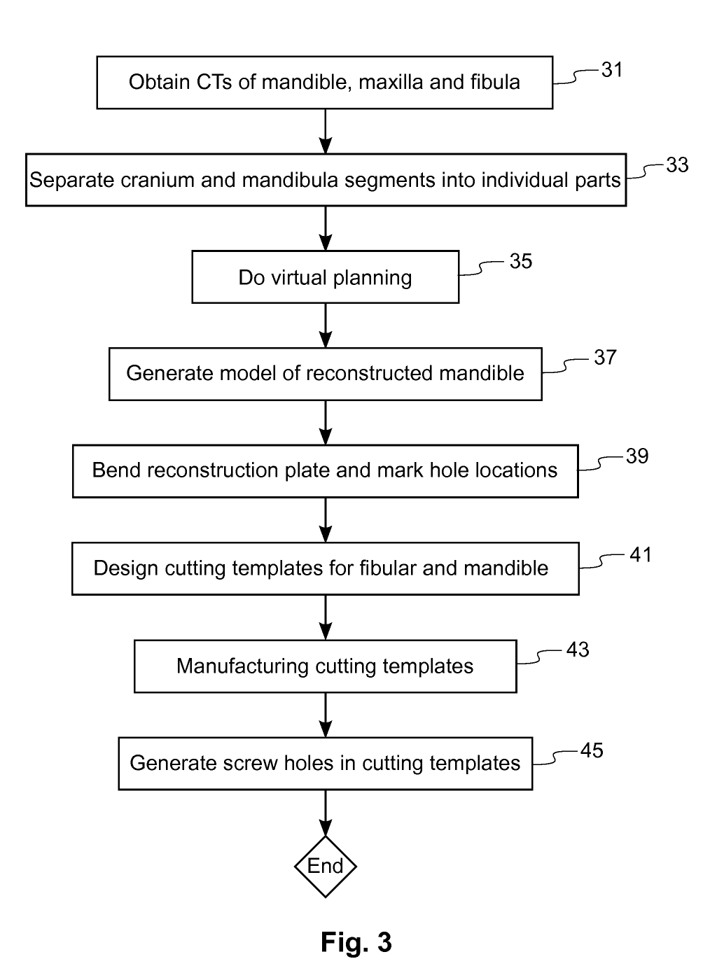

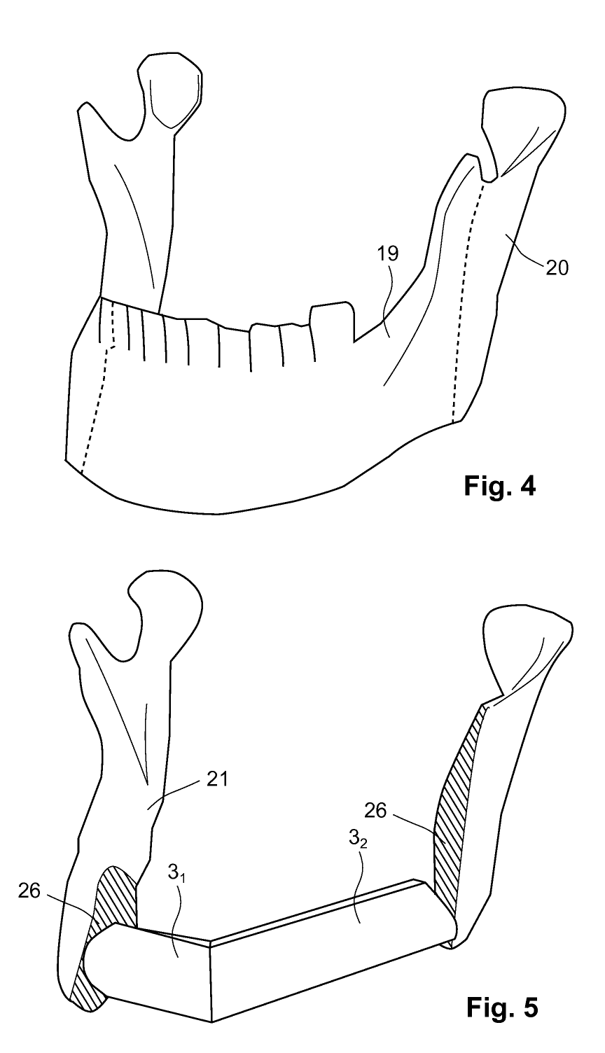

Next, the method of manufacturing the cutting template 1′ is described with reference to the figures. The flow chart of FIG. 3 summarises the manufacturing method steps. In step 31, computer topographies (CTs) of the bony mandible and maxilla (facial scan), and the fibula (possibly with concomitant CT-angiography) are carried out. In this manner, preoperative 3-dimensional visual reconstruction of the mandible and the osteocutaneous fibula free flap can be obtained. The CT in this example uses a 1.0-mm slice thickness CT operation. In step 33, the preoperative CT scans are imported into a segmentation or slicer software application to create digital volumetric models for surgical planning. The cranium and the mandibular fragments are separated into individual parts, and the application generates a 3-dimensional surface of the regions of interest. In step 35, virtual planning is carried out. In order to do this, the output data from the segmentation application is imported into a design software application, in this example a 3D computer-aided open source software application. The data may be imported for example in a stereolithography (STL) file format. The virtual planning may comprise performing positron emission tomography (PET) and magnetic resonance imaging (MRI) scans to define resection locations (coordinates) and margins with safety distances. FIG. 4 shows an example result of such modelling operations. The portion 19 of the mandible 20 which is to be cut away is the portion between the dashed lines in FIG. 4. Simulations of various procedures, such as cutting, interpositioning and rotation of the bone segments may also be carried out in this step. The arrangement of the fibula segments and their separation into aligned sections may be done in close collaboration with a prosthetic dentist to achieve an ideal mandibular-maxillary relation for the later dental rehabilitation. FIG. 5 shows a simulation where two aligned fibula sections 3₁, 3₂ have been inserted in place of the cut-away portion 19 of the mandible.

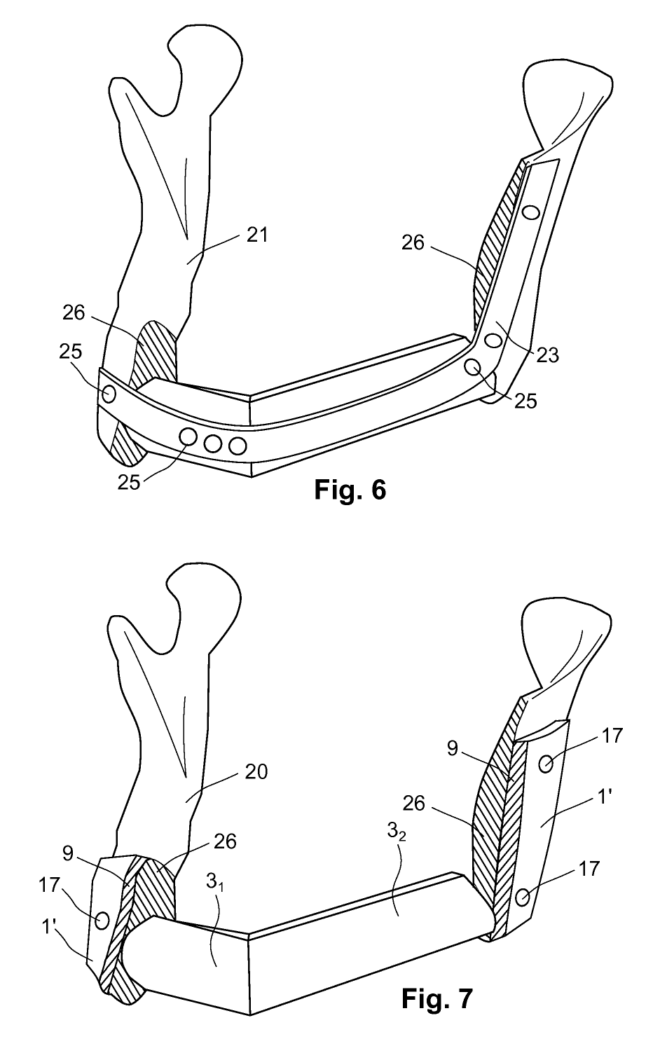

Once the virtual surgery has been finished, in step 37 a physical model 21 of the reconstructed mandible is generated. This is preferably carried out by 3-dimensional printing. In step 39, with the aid of this physical model, a conventional bridging reconstruction plate 23 is pre-bent manually by the operator, ie the surgeon, by fitting it to the physical model. The reconstruction plate 23 fitted on to the generated model 21 is shown in FIG. 6. The reconstruction plate has through-holes 25 for bone screws to pass through for fixing the reconstruction plate to the bone (in this case eventually to the reconstructed mandible). By using the reconstruction plate 23, the locations of the screw holes are then marked on the model 21. It is also possible to make real through holes in the model 21 at the locations indicated by the holes 25 in the reconstruction plate 23.

It is to be noted that in the reconstruction of the mandible, in cases of extensive bony defects, and particularly when a large interior area and the body of the mandible are missing, it would be difficult without the approach above, to place the reconstruction plate 23 correctly and obtain the desired position when no anatomical orientation is available. If the plate 23 is bent after the mandibular resection, the only reference point is the positional relationship to the maxilla and maxillary teeth. Also, the remaining proximal fragments are in an unstable position, and it is extremely difficult to know the exact 3-dimensional configuration of the mandibular defect. For this reason, in the proposed method, in cases of pre-existing bony defects, the healthy side of the mandible should be realigned to a proper dental occlusion and then be mirrored over the middle plane to simulate the original shape of the mandible 20. In addition, the plate is properly bent before the mandibular resection by relying on the virtual planning so as to achieve a natural and firm contour of the reconstructed mandible, without any anatomical disorientations.

In step 41, the cutting templates 1, 1′ are designed and created for fibular and mandibular osteotomies. In this example, separate cutting templates 1, 1′ are manufactured for fibular and mandibular osteotomies so that that the template for mandible comprise two cutting guides, while the template for the fibular comprises four cutting guides, as shown in FIGS. 1 and 7. These cutting templates 1, 1′ are designed with a computer, using for example the design software application. In the design process, scan data from a region where the bone is to be cut is used to perform the steps of:
a) determining 3D coordinates of at least two cuts to be performed in the bone,
b) determining 3D coordinates of the cutting guides 9, 9′ for performing the cuts,
c) determining 3D coordinates of the bone surface in the region of the cuts and/or the positioning elements 13.

In step 43 the cutting templates are manufactured based on the scan data and the 3D coordinates determined in the above steps a), b) and c). This can be done for example by 3-dimensional printing. The 3D coordinates determined in step c) are used to shape the bone mating surfaces to mate with the bone surface in the vicinity of the cuts and/or the positioning elements 13. Then the mandibular cutting template 1′ is fitted to the printed mandibular model 21 and, in step 45, holes 17′ are generated in the mandibular cutting template 1′ according the marks on the model 21. This has the advantage that the reconstruction plate 23 can then later be fixed to the reconstructed mandible at the locations of these holes 17 (when the cutting template is in the cutting position). At the same time the holes 17 can be generated also in the fibular cutting template 1.

A method of manufacturing the cutting templates 1, 1′ was described above. A method of using the cutting templates 1, 1′ is briefly described next. Once the bones in the regions where the cutting templates 1, 1′ are to be placed for bone cutting are deperiostated, the surgeon places the cutting template 1′ on the mandible, and drills bone screws through the holes 17 in the cutting template, and fixes the template with the screws to the mandible of the patient. Next, the surgeon performs a mandibular bone cutting operation by using the cutting guides 9′ to precisely guide the saw. Once this has been done, the cutting template 1′ is removed. Then, the sterilised pre-bent reconstruction plate 23 is fixed in the predetermined position with screws through holes in the mandible, which were made earlier.

At the same time, another surgical team works with the fibular free flap. Again the deperiostation is performed in the regions where the fibular osteotomy is to be carried out. Once the deperiostation has been completed, the fibular cutting template 1 is fixed with bone screws to the patients fibular. Next, the actual bone cutting operation can be carried out in a similar manner to the cutting of the mandible. In this example, two separate sections or portions 3₁, 3₂ of the fibular are cut, and they are placed at the location where the mandibular portion 19 was removed. These two replacement portions 3₁, 3₂ are carefully aligned and fixed to the reconstruction plate 23 with bone screws. The replacement portions 3₁, 3₂ are also carefully mated with the stump surfaces 26 of the mandible 20 (see FIG. 6). To finalise the operation, proper vascular anastomoses are performed, and the wound is then closed carefully. Postoperatively, multi-slice CT scans may be taken for control purposes. The preoperative shape of the mandible, including the graft position, may be compared with the postoperative result by virtually overlaying the two datasets. By using the proposed cutting templates 1, 1′, an easy, precise and highly flexible mandible reconstruction can be carried out with the fibula free flap.

While the invention has been illustrated and described in detail in the drawings and foregoing description, such illustration and description are to be considered illustrative or exemplary and not restrictive, the invention being not limited to the disclosed embodiment. Other embodiments and variants are understood, and can be achieved by those skilled in the art when carrying out the claimed invention, based on a study of the drawings, the disclosure and the appended claims.

In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A cutting template for mandibular surgery, comprising:
at least two cutting guides for guiding a bone cutting element for cutting a bone,
at least one opening for receiving bone fixation means for fixing the cutting template to the bone, and
at least one positioning element for positioning the cutting template against the bone, wherein the at least one positioning element comprises a first bone mating surface shaped with a first surface contour of the bone, for intimate contact with the bone during cutting.

2. The cutting template according to claim 1, wherein at least one of the at least two cutting guides is arranged for being in intimate contact with the bone during cutting.

3. The cutting template according to claim 1, wherein the at least one of the at least two cutting guides comprises a second bone mating surface shaped with a second surface contour of the bone.

4. The cutting template according to claim 1, wherein the cutting template further comprises a base supporting the at least two cutting guides.

5. The cutting template according to claim 4, wherein the base has a substantially straight or curved shape or comprises at least two substantially straight sections having a predetermined angle between them.

6. The cutting template according to claim 4, wherein the base and the cutting guides are substantially arranged in a common plane.

7. The cutting template according to claim 1, wherein the at least two cutting guides each comprises a longitudinal cutting slot for receiving the bone cutting element, and wherein the cutting slot defines a cutting plane.

8. The cutting template according to claim 7, wherein at least some of the cutting planes are not parallel to one another.

9. The cutting template according to claim 7, wherein the surface of the base from which the cutting guides protrude defines a base surface plane, and wherein at least one cutting plane is substantially orthogonal to the base surface plane.

10. The cutting template according to claim 1, wherein the cutting template comprises two or four cutting guides.

11. The cutting template according to claim 1, wherein the positioning element comprises the at least one opening.

12. A method of manufacturing the cutting template according to claim 1, the method comprising using scan data from a region where the bone is to be cut to perform the steps of:
a) determining 3D coordinates of at least two cuts to be performed in the bone,
b) determining 3D coordinates of the cutting guides for performing the cuts,
c) determining 3D coordinates of the bone surface in the region of the cuts,
d) manufacturing the cutting template, and
e) using the 3D coordinates determined in step c) to shape at least the first bone mating surface to mate with the bone surface in the vicinity of at least one of the cuts.

13. The method of manufacturing the cutting template according to claim 12, wherein the manufacturing of the cutting template comprises using 3D printing for generating the cutting template.

14. The method of manufacturing the cutting template according to claim 12, wherein the openings are generated at locations indicated by holes in a reconstructed bone model.

15. A computer program product comprising instructions for implementing the steps of the method according to claim 12 when loaded and run on computer means of an electronic device.
